(19) United States
(12) Patent Application Publication
(10) Pub. No.: US 2024/0382657 A1
(43) Pub. Date: Nov. 21, 2024

(54) SYSTEM AND METHOD FOR SYRINGE PUMP BLOOD TRANSFUSION

(71) Applicant: CAREFUSION 303, INC., San Diego, CA (US)

(72) Inventors: Vishnu MALLAPRAGADA, San Diego, CA (US); Janice R. PAK, Centennial, CO (US)

(21) Appl. No.: 18/691,866

(22) PCT Filed: Sep. 13, 2022
(86) PCT No.: PCT/US2022/043381

§ 371 Date: Mar. 13, 2024

Related U.S. Application Data
(60) Provisional application No. 63/244,209, filed on Sep. 14, 2021.

(51) Int. Cl. A61M 1/02; A61M 5/145; A61M 39/24

(52) U.S. Cl. CPC A61M 1/024; A61M 1/0272; A61M 5/1452; A61M 39/24; A61M 2205/3331; A61M 2205/52

(57) ABSTRACT

A method for transfusing blood using a syringe pump is disclosed. The method includes connecting a blood supply line to an upstream connection of a check valve, an intravenous transfusion line to a downstream connection of the check valve, and a syringe pump to a second upstream connection of the check valve, and activating the syringe pump to automatically retract and drive a plunger of the syringe pump, repeatedly, according to a pump rate programmed into the syringe pump according to the automatic retracting and driving of the plunger. By this mechanism, a blood product is automatically withdrawn from the blood supply line, through the check valve, and into a chamber of a syringe loaded into the syringe pump during each cycle of the automatic retracting of the plunger, and provided through the intravenous transfusion line from the chamber during each cycle of the automatic driving of the plunger.

### BACKGROUND

This application relates generally to automated blood transfusions. Delivering large amounts of blood over a small period of time, such as in trauma situations, may include use of an in-line hand pump, or in some cases pressure cuffs around the blood intravenous (IV) container (e.g., bag) that are also manually pumped operated. This is ergonomically not ideal and results in fatigue for the clinician. It may not always be the most efficient method for trying to increase the flow of blood to the patient as in many cases the hand pump causes back flow of blood as the hand pump refills with blood with each pump cycle.

### SUMMARY

This subject technology provides an IV set designed for large volume delivery of fluids at high flow rates, particularly addressing the delivery of large volumes of blood in trauma situations. The IV set is designed to be used with a syringe pump and includes a double check valve as an interface to the syringe pump (e.g., upstream and downstream check valve operation). During the fill phase, the linear actuator retracts the syringe to draw in fluid from the IV bag. The downstream back check valve prevents fluid draw from the patient side, while allowing fluid to be drawn from the infusion container (bag/bottle). During the delivery phase, the linear actuator extends to deliver fluid to the patient. The upstream back check valve prevents back flow to the IV bag while allowing flow to the patient. The linear actuator may be instrumented with an encoder and pressure sensor to control flow rate and monitor for occlusions.

According to various aspects of the subject technology, a method for transfusing blood using a syringe pump is disclosed. The method includes connecting a blood supply line to an upstream connection of a check valve, connecting an intravenous transfusion line to a downstream connection of the check valve, connecting a first syringe pump to a second upstream connection of the check valve, and activating the syringe pump to automatically retract and drive a plunger of the first syringe pump, repeatedly, according to a pump rate programmed into the first syringe pump to produce respective negative and positive pressures within the check valve according to the automatic retracting and driving of the plunger, whereby a blood product is withdrawn from the blood supply line, through the upstream connection of the check valve, and into a chamber of a syringe loaded into the first syringe pump during each cycle of the automatic retracting of the plunger, and provided through the intravenous transfusion line from the chamber during each cycle of the automatic driving of the plunger. Other aspects include corresponding systems, apparatus, and computer program products for implementation of the corresponding method and its features.

According to various implementations, the method further includes connecting a drip chamber to the blood supply line, and connecting a blood product container to the drip chamber (including, for example, an anti-run dry (ARD) membrane), wherein the blood product container supplies a blood product to the blood supply line via the drip chamber. In some implementations, the method further includes connecting a flow control element to the blood supply line, and regulating a flow of the blood product through the blood supply line using the flow control element. In some implementations, the method further includes connecting a pressure sensor at a location on the intravenous transfusion line downstream of the check valve, monitoring, with the pressure sensor, a real-time delivery pressure associated with flow of the blood product, and automatically adjusting the pump rate based on the real-time delivery pressure.

The use of a syringe pump to facilitate transfusion results in delivering blood quickly to the patient and eliminates the risk of a clinician falling into fatigue and unable to continue to pump the blood to increase flow (e.g., by a hand mechanism). Additionally, the subject technologies use of an in-line double check valve prevents back flow of blood away from the patient during each pump cycle. Other elements of the IV set design such as the addition of an anti-run dry (ARD) membrane to keep the line primed when the blood bag must be replaced and specifying a larger ID tubing to allow for a higher flow rate, will also make this dedicated pump set effective for quick and constant delivery of blood and/or other fluids in urgent situations. The addition of the encoder and pressure sensor in the syringe pump also ensures accurate delivery of the IV fluid/blood.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example of an institutional patient care system of a healthcare organization, according to aspects of the subject technology.

FIG. 2 shows an example syringe pump infusion device, according to various aspects of the subject technology.

FIGS. 3A and 3B depict an example transfusion system coupled to a patient, according to various aspects of the subject technology.

FIG. 4 depicts a second example transfusion system coupled to a patient, including two syringe pumps, according to various aspects of the subject technology.

FIG. 5 depicts an example process for transfusing blood using a syringe pump, according to aspects of the subject technology.

FIG. 6 is a conceptual diagram illustrating an example electronic system for transfusing blood using a syringe pump, according to aspects of the subject technology.

### DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

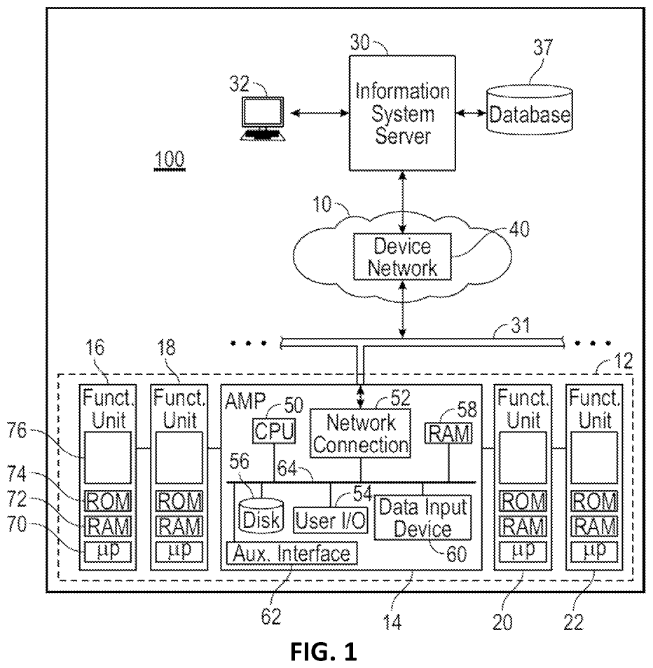

FIG. 1 depicts an example of an institutional patient care system 100 of a healthcare organization, according to aspects of the subject technology. In FIG. 1, a patient care device (or “medical device” generally) 12 is connected to a hospital network 10. The term patient care device (or “PCD”) may be used interchangeably with the term patient care unit (or “PCU”), either which may include various ancillary medical devices such as an infusion pump, a vital signs monitor, a medication dispensing device (e.g., cabinet, tote), a medication preparation device, an automated dispensing device, a module coupled with one of the aforementioned (e.g., a syringe pump module configured to attach to an infusion pump), or other similar devices. Each element 12 is connected to an internal healthcare network 10 by a transmission channel 31. Transmission channel 31 is any wired or wireless transmission channel, for example an 802.11 wireless local area network (LAN). In some implementations, network 10 also includes computer systems located in various departments throughout a hospital. For example, network 10 of FIG. 1 optionally includes computer systems associated with an admissions department, a billing department, a biomedical engineering department, a clinical laboratory, a central supply department, one or more unit station computers and/or a medical decision support system. As described further below, network 10 may include discrete subnetworks. In the depicted example, network 10 includes a device network 41 by which patient care devices 12 (and other devices) communicate in accordance with normal operations.

Additionally, institutional patient care system 100 may incorporate a separate information system server 130, the function of which will be described in more detail below. Moreover, although the information system server 130 is shown as a separate server, the functions and programming of the information system server 130 may be incorporated into another computer, if such is desired by engineers designing the institution's information system. Institutional patient care system 100 may further include one or multiple device terminals 132 for connecting and communicating with information system server 130. Device terminals 132 may include personal computers, personal data assistances, mobile devices such as laptops, tablet computers, augmented reality devices, or smartphones, configured with software for communications with information system server 130 via network 10.

Patient care device 12 comprises a system for providing patient care, such as that described in Eggers et al., which is incorporated herein by reference for that purpose. Patient care device 12 may include or incorporate pumps, physiological monitors (e.g., heart rate, blood pressure, ECG, EEG, pulse oximeter, and other patient monitors), therapy devices, and other drug delivery devices may be utilized according to the teachings set forth herein. In the depicted example, patient care device 12 comprises a control module 14, also referred to as interface unit 14, connected to one or more functional modules 116, 118, 120, 122. Interface unit 14 includes a central processing unit (CPU) 50 connected to a memory, for example, random access memory (RAM) 58, and one or more interface devices such as user interface device 54, a coded data input device 60, a network connection 52, and an auxiliary interface 62 for communicating with additional modules or devices. Interface unit 14 also, although not necessarily, includes a main non-volatile storage unit 56, such as a hard disk drive or non-volatile flash memory, for storing software and data and one or more internal buses 64 for interconnecting the aforementioned elements.

In various implementations, user interface device 54 is a touch screen for displaying information to a user and allowing a user to input information by touching defined areas of the screen. Additionally, or in the alternative, user interface device 54 could include any means for displaying and inputting information, such as a monitor, a printer, a keyboard, softkeys, a mouse, a track ball and/or a light pen. Data input device 60 may be a bar code reader capable of scanning and interpreting data printed in bar coded format. Additionally or in the alternative, data input device 60 can be any device for entering coded data into a computer, such as a device(s) for reading a magnetic strips, radio-frequency identification (RFID) devices whereby digital data encoded in RFID tags or smart labels (defined below) are captured by the reader 60 via radio waves, PCMCIA smart cards, radio frequency cards, memory sticks, CDs, DVDs, or any other analog or digital storage media. Other examples of data input device 60 include a voice activation or recognition device or a portable personal data assistant (PDA). Depending upon the types of interface devices used, user interface device 54 and data input device 60 may be the same device. Although data input device 60 is shown in FIG. 1C to be disposed within interface unit 14, it is recognized that data input device 60 may be integral within pharmacy system 34 or located externally and communicating with pharmacy system 34 through an RS-232 serial interface or any other appropriate communication means. Auxiliary interface 62 may be an RS-232 communications interface, however any other means for communicating with a peripheral device such as a printer, patient monitor, infusion pump or other medical device may be used without departing from the subject technology. Additionally, data input device 60 may be a separate functional module, such as modules 116, 118, 120 and 122, and configured to communicate with controller 14, or any other system on the network, using suitable programming and communication protocols.

Network connection 52 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

Functional modules 116, 118, 120, 122 are any devices for providing care to a patient or for monitoring patient condition. As shown in FIG. 1C, at least one of functional modules 116, 118, 120, 122 may be an infusion pump module such as an intravenous infusion pump for delivering medication or other fluid to a patient. For the purposes of this discussion, functional module 116 is an infusion pump module. Each of functional modules 118, 120, 122 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Functional module 118, 120 and/or 122 may be a printer, scanner, bar code reader, near-field communication reader, RFID reader, or any other peripheral input, output or input/output device.

Each functional module 116, 118, 120, 122 communicates directly or indirectly with interface unit 14, with interface unit 14 providing overall monitoring and control of device 12. Functional modules 116, 118, 120, 122 may be connected physically and electronically in serial fashion to one or both ends of interface unit 14 as shown in FIG. 1C, or as detailed in Eggers et al. However, it is recognized that there are other means for connecting functional modules with the interface unit that may be utilized without departing from the subject technology. It will also be appreciated that devices such as pumps or patient monitoring devices that provide sufficient programmability and connectivity may be capable of operating as stand-alone devices and may communicate directly with the network without connected through a separate interface unit or control unit 14. As described above, additional medical devices or peripheral devices may be connected to patient care device 12 through one or more auxiliary interfaces 62.

Each functional module 116, 118, 120, 122 may include module-specific components 76, a microprocessor 70, a volatile memory 72 and a nonvolatile memory 74 for storing information. It should be noted that while four functional modules are shown in FIG. 1C, any number of devices may be connected directly or indirectly to central controller 14. The number and type of functional modules described herein are intended to be illustrative, and in no way limit the scope of the subject technology. Module-specific components 76 include any components necessary for operation of a particular module, such as a pumping mechanism for infusion pump module 116.

While each functional module may be capable of at least some level of independent operation, interface unit 14 monitors and controls overall operation of device 12. For example, as will be described in more detail below, interface unit 14 provides programming instructions to the functional modules 116, 118, 120, 122 and monitors the status of each module.

Patient care device 12 is capable of operating in several different modes, or personalities, with each personality defined by a configuration database. The configuration database may be a database 56 internal to patient care device, or an external database 37. A particular configuration database is selected based, at least in part, by patient-specific information such as patient location, age, physical characteristics, or medical characteristics. Medical characteristics include, but are not limited to, patient diagnosis, treatment prescription, medical history, medical records, patient care provider identification, physiological characteristics or psychological characteristics. As used herein, patient-specific information also includes care provider information (e.g., physician identification) or a patient care device's 10 location in the hospital or hospital computer network. Patient care information may be entered through interface device 52, 54, 60 or 62, and may originate from anywhere in network 10, such as, for example, from a pharmacy server, admissions server, laboratory server, and the like.

Medical devices incorporating aspects of the subject technology may be equipped with a Network Interface Module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments, and such environments are intended to be within the scope of the subject technology.

Data to and from the various data sources can be converted into network-compatible data with existing technology, and movement of the information between the medical device and network can be accomplished by a variety of means. For example, patient care device 12 and network 10 may communicate via automated interaction, manual interaction or a combination of both automated and manual interaction. Automated interaction may be continuous or intermittent and may occur through direct network connection 54 (as shown in FIG. 1), or through RS232 links, MIB systems, RF links such as BLUETOOTH, IR links, WLANS, digital cable systems, telephone modems or other wired or wireless communication means. Manual interaction between patient care device 12 and network 10 involves physically transferring, intermittently or periodically, data between systems using, for example, user interface device 54, coded data input device 60, bar codes, computer disks, portable data assistants, memory cards, or any other media for storing data. The communication means in various aspects is bidirectional with access to data from as many points of the distributed data sources as possible. Decision-making can occur at a variety of places within network 10. For example, and not by way of limitation, decisions can be made in health information system (HIS) server 30, decision support 48, remote data server 49, hospital department or unit stations 46, or within patient care device 12 itself.

All direct communications with medical devices operating on a network in accordance with the subject technology may be performed through information system server 30, known as the remote data server (RDS). In accordance with aspects of the subject technology, network interface modules incorporated into medical devices such as, for example, infusion pumps or vital signs measurement devices, ignore all network traffic that does not originate from an authenticated RDS. The primary responsibilities of the RDS of the subject technology are to track the location and status of all networked medical devices that have NIMs, and maintain open communication.

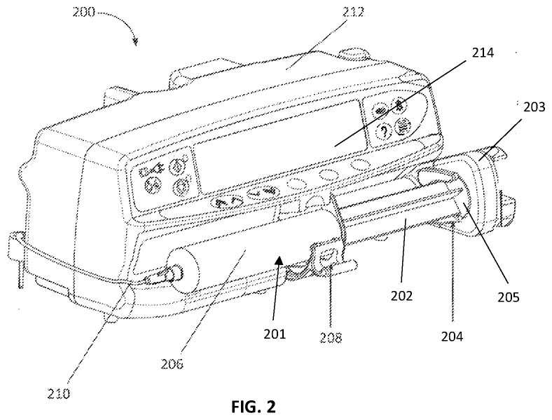

FIG. 2 shows an example syringe pump 200 infusion device, according to various aspects of the subject technology. While the example syringe pump 200 is shown as a stand alone device, the syringe pump may be configured as a functional module 16, 18, 20, 22 for operable connection to and/or coupling with a control unit 14 of the previously described medical device.

The syringe pump is configured to load a disposable syringe 206 for administration of a fluid. When a syringe 201 is mounted in the syringe pump 200 properly, a plunger flange at the end of the syringe piston 202 is held in a plunger drive head 203 with a pair of pivotally mounted plunger retaining arms. The syringe 206 is secured by a syringe clamp 208. The drive head 203 includes a pushing surface on which the plunger flange will rest as the drive head 203 moves forward toward the plunger barrel 206 pushing the plunger piston 202 into the barrel 206 of the syringe to expel the syringe contents through an administration tubing 210 to the patient. The drive head may be connected to a screw drive mechanism, including a motor, for connecting the linear motion of the screw drive mechanism to the syringe plunger in order to empty the syringe. The rate is controlled by the syringe pump 200 based on the programmed parameter (e.g., desired rate) and type of syringe.

According to various implementations, the syringe 206 holds a medical fluid to be infused by the syringe pump 200. As described herein, the syringe 206 may be of a make and mode configured to be compatible with a blood product. For the purpose of this disclosure, a blood product, or fluid, may include whole blood, blood components, and/or plasma derivatives. Blood components may include red blood cell concentrates or suspensions, platelets, plasma, and/or cryoprecipitate. Plasma derivatives may include plasma proteins prepared under pharmaceutical manufacturing conditions, such as albumin, coagulation factor concentrates, and/or immunoglobulins.

Syringe pumps do not typically experience any upstream pressure conditions because the fluid to be infused is housed in the syringe 206 and is pushed into an administration set 210 by way of the plunger 202. Downstream pressure conditions can be detected by a force sensor housed in or upon a pump system 212 according to the methods described here, which are readily applied to syringe pumps. The force sensor measures the force exerted by the drive head 204 of the syringe pump on the syringe plunger 202.

Syringe pumps do not typically experience any upstream pressure conditions because the fluid to be infused is housed in the syringe barrel 206 and is pushed into an administration set 210 by way of the plunger 202. Downstream pressure conditions can be detected by a force sensor housed in or upon a pump system 212 according to the methods described here, which are readily applied to syringe pumps. The force sensor measures the force exerted by the drive head 203 of the syringe pump on the syringe piston 202.

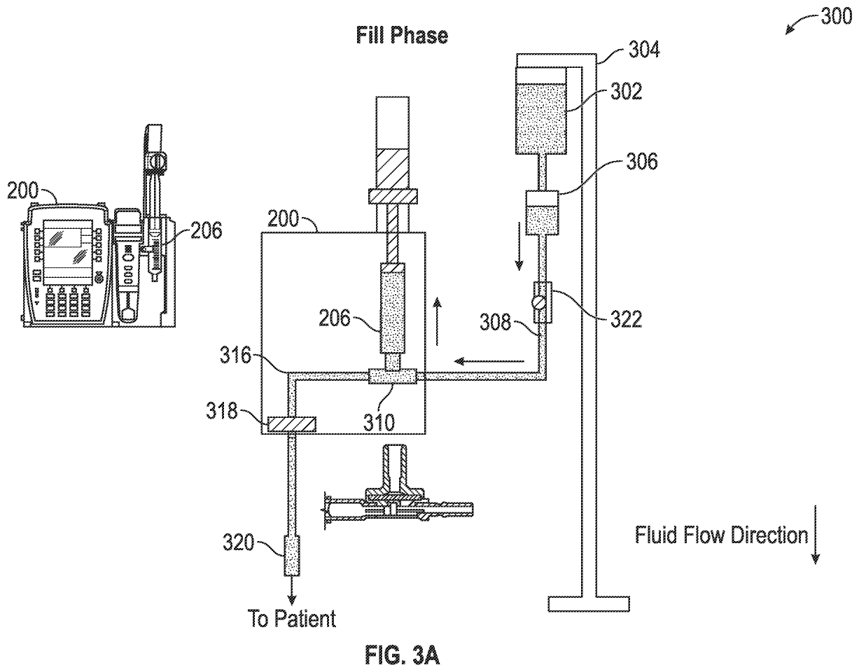

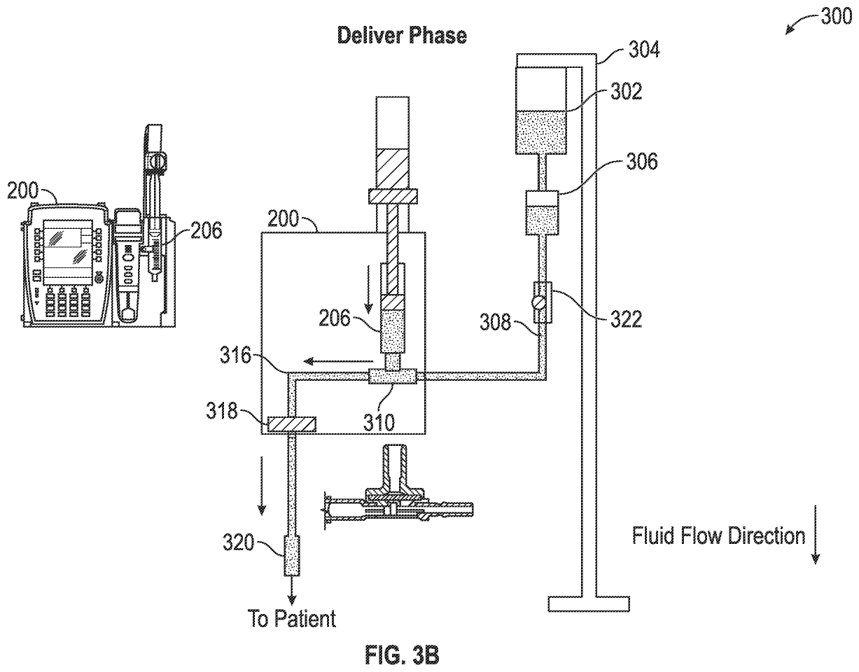

In some embodiments, the syringe pump may include a high resolution pressure sensor that interfaces with a pressure disc 318 (FIG. 3) on the syringe administration set. The pressure disc provides a relatively large area in contact with the pressure sensor. This allows the pressure sensor to measure the pressure inside the administration set more directly (not through the syringe plunger head) and with higher resolution and higher accuracy compared with the drive head force sensor. The measurements from this pressure sensor and the drive head force sensor can be used independently or in conjunction with each other to detect an empty condition in a syringe pump.

In an infusion pump, various components that lie in an infusion path such as administration set, cannula, filters, and valves exhibit both resistance and compliance. In normal operation, the pump generates a pressure, termed a working pressure, to overcome the resistance of these and other components in the infusion path.

As well as operating buttons or switches, which the operator may use to activate and program the syringe pump 200, there is a display screen 214. The display screen 214 may be a simple LCD (liquid crystal display) having a small number of segments, for example seven segments in a figure-of-eight configuration per character, adapted to display a small number of alphanumeric characters. The display may be monochromatic, for example, it might only display red, green or grey/black characters. Alternatively, the display 214 might be a more complicated liquid crystal display capable of displaying more characters or more complicated characters. The LCD may be backlit, for example, using light emitting diodes (LEDs). In some implementations, the infusion pump may include a TFT LCD. A TFT is a thin-film transistor-based LCD technology. In some implementations, the display screen 214 is also a touchscreen such as a capacititive touchscreen.

When programming an infusion device, the user must input the type of syringe being fitted to the pump. The pump stores in an internal memory a database of known syringe types containing information such as syringe diameter and stroke. The infusion pump firmware calculates the position of the syringe plunger and syringe piston based on movement of the syringe driver head and the type and size of the syringe. This allows the machine to display the calculation of volume infused, time elapsed, volume remaining and time remaining. As infusion continues and the driver head moves, these calculations can be updated, and the displayed information changed.

Infusion pump may be provided with a scrolling system comprising an “up” scroll key and a “down” scroll key which are operable to increase or decrease pumping parameters, such as the mass flow rate setting shown on a display, or the VTBI (volume to be infused) setting shown on the display. In some cases, each scroll key may be physically present on the device (as depicted), or may be graphically displayed in a touchscreen display 214.

FIGS. 3A and 3B depict an example transfusion system 300 coupled to a patient, according to various aspects of the subject technology. FIG. 3A depicts the transfusion system 300 during a fill phase, and FIG. 3B depicts the transfusion system during a delivery phase. In particular, a fluid source 302 containing an intravenous (IV) fluid is held on an intravenous pole 304. According to various implementations, the fluid source is an IV bag or blood product bag. A drip chamber 306 is coupled to an outlet of the fluid source 302, and an upstream portion of the depicted infusion line 308 is connected to an outlet of the drip chamber 306. The upstream infusion line 308 may be a conventional IV infusion-type tube typically used in a hospital or medical environment, and is made of any type of flexible tubing appropriate for use to infuse therapeutic fluids into a patient, such as polyvinylchloride (PVC).

The upstream infusion line 308 is connected to a double check valve 310 at a first upstream connection point 312 of the valve. A downstream connection 314 of the double check valve 310 is connected to a flexible downstream infusion line 316, which is then connected to a patient. A pressure sensor 318 (e.g., a sensing disc or force sensor) may be located at a portion of the downstream infusion line 316 to sense a pressure within downstream infusion line 316. According to various aspects, downstream infusion line 316 may terminate at a luer connection 320 configured to attach to a patient infusion set (not shown) for administration of a fluid from the fluid source directly into a vein of the patient, for example in the patient's arm.

A syringe pump 200 is shown connected at a second upstream connection 320 of the check valve 310. In this regard, syringe pump 200 may retract to cause a negative pressure in the check valve 310, drawing the fluid of the fluid source 302 from the drip chamber 306, through upstream infusion line 308, and through the check valve 310 and into the downstream infusion line 316 for administration to the patient. According to various aspects, as the syringe plunger 202 is retracted, the fluid may at least partially fill the syringe chamber 206, and then be expelled when the syringe is pushed forward by way of the plunger. A first upstream valve of the double check valve prevents fluid drawn into the check valve by the syringe, or expelled from the syringe, from backflowing into the upper infusion line 308. A downstream back check valve of the double check valve prevents fluid draw from the patient side.

According to some implementations, upstream infusion line 308 may include a flow control element 322 (e.g., a roller clamp or flow controller) to control the rate at which the fluid within drip chamber 306 is provided by the upstream infusion line 308 to the check valve 310 and downstream infusion line 316. The pump may include a linear actuator to actuate the plunger 206. The linear actuator may be instrumented with an encoder (not shown) operating in connection with the pressure sensor 318 to control flow rate and monitor for occlusions.

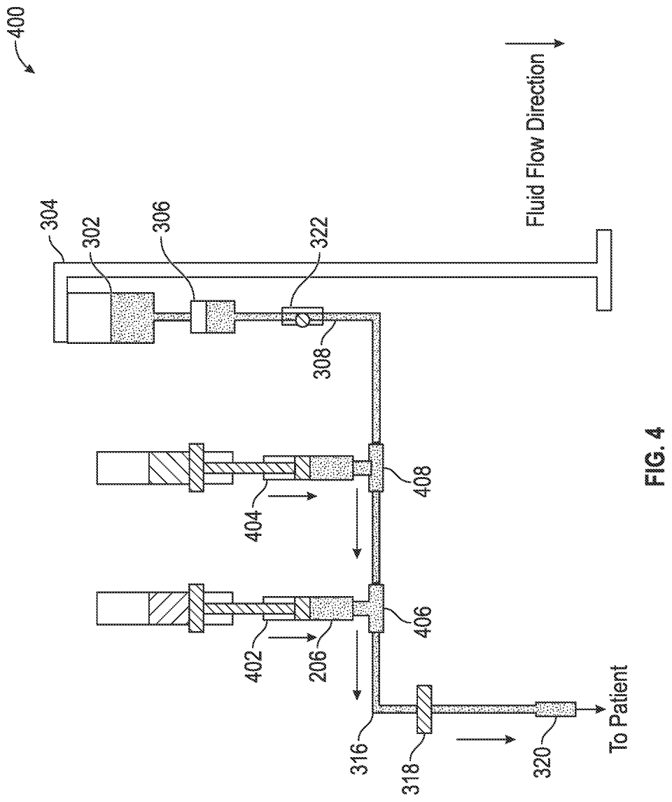

FIG. 4 depicts a second example transfusion system 400 coupled to a patient, including two syringe pumps 402, 404, according to various aspects of the subject technology. As depicted by FIG. 4, two check valves 406, 408 may be connected to the blood supply line, upstream of an intravenous transfusion line, and a second syringe pump connected to an upstream connection of the second check valve. The syringe pumps may be separate units or modules (16, 18, 20, 22 of FIG. 1) of a single infusion device 12. For example, as depicted in FIG. 1, the first and second syringe pumps may be removably connected to a pump control unit 14 by way of respective plugin ports on the control unit. In this regard, the control unit includes a processor and the processor communicates with the first and second syringe pumps via electrical signals communicated through the respective plug in ports.

The first syringe pump 402 may be activated to automatically retract and drive a plunger of the first syringe pump, repeatedly, according to a pump rate programmed into the first syringe pump to produce respective negative and positive pressures within the check valve according to the automatic retracting and driving of the plunger. The second syringe pump 404 may also be activate, for example, in coordination with the first syringe pump, to automatically retract and drive a plunger of the second syringe pump, repeatedly. In this manner, the first and second syringe pumps may be instructed by the processor to retract and drive their respective plungers according to respective timing patterns offset from each other to deliver the blood product to the intravenous transfusion line.

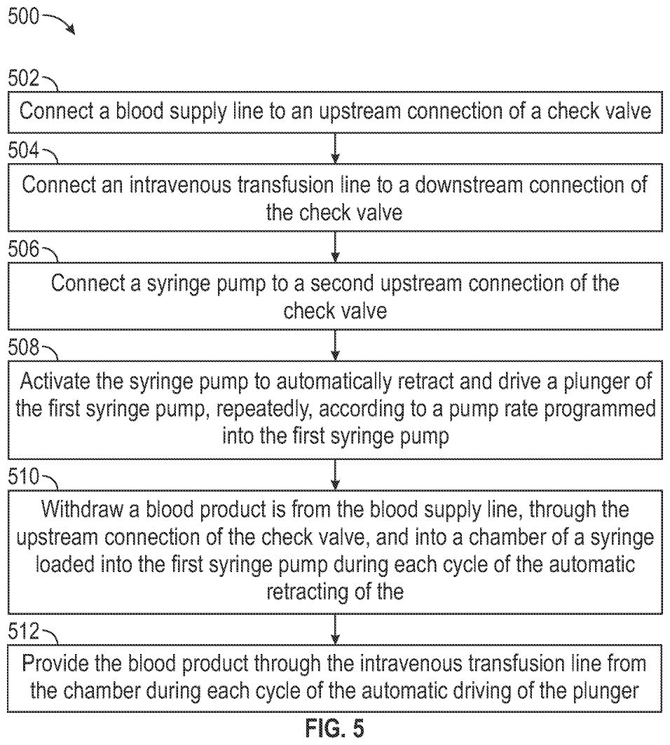

FIG. 5 depicts an example process for transfusing blood using a syringe pump, according to aspects of the subject technology. For explanatory purposes, the various blocks of example process 200 are described herein with reference to FIGS. 1-4, and the components and/or processes described herein. The one or more of the blocks of process 500 may be implemented, for example, by one or more computing devices including, for example, medical device 12 and/or syringe pump 200. In some implementations, one or more of the blocks may be implemented based on one or more machine learning algorithms. In some implementations, one or more of the blocks may be implemented apart from other blocks, and by one or more different processors or devices. Further for explanatory purposes, the blocks of example process 500 are described as occurring in serial, or linearly. However, multiple blocks of example process 500 may occur in parallel. In addition, the blocks of example process 500 need not be performed in the order shown and/or one or more of the blocks of example process 500 need not be performed.

In the depicted example, a blood supply line is connected to an upstream connection of a check valve 310 (502). An intravenous transfusion line is connected to a downstream connection of the check valve 310 (504). A syringe pump 200 is connected to a second upstream connection of the check valve 310 (506).

The syringe pump 200 is activated to automatically retract and drive a plunger of the first syringe pump, repeatedly, according to a pump rate programmed into the first syringe pump to produce respective negative and positive pressures within the check valve according to the automatic retracting and driving of the plunger (508). By this mechanism, a blood product is withdrawn from the blood supply line, through the upstream connection of the check valve, and into a chamber of a syringe loaded into the first syringe pump during each cycle of the automatic retracting of the plunger (510), and provided through the intravenous transfusion line from the chamber during each cycle of the automatic driving of the plunger (512).

According to various implementations, a blood product container 302 supplies the blood product to the blood supply line. In some implementations, as depicted in FIGS. 3A and 3B, a drip chamber may be connected to the blood supply line, and the blood product container to the drip chamber. In this regard, the blood product container 302 supplies a blood product to the blood supply line via the drip chamber.

In some implementations, a flow control element 322 may be connected to the blood supply line and a flow of the blood product through the blood supply line controlled or regulated using the flow control element. In some implementations, a pressure sensor 318 may be connected at a location on the intravenous transfusion line 316 downstream of the check valve 310. A real-time delivery pressure associated with flow of the blood product may then be monitored by the pressure sensor. The syringe pump 200 may provide the monitoring and automatically adjust the pump rate based on the real-time delivery pressure. In some implementations, adjusting the pump rate may include increasing a speed at which a plunger of the syringe moves to provide the blood product from the syringe.

Many of the above-described example process 600, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

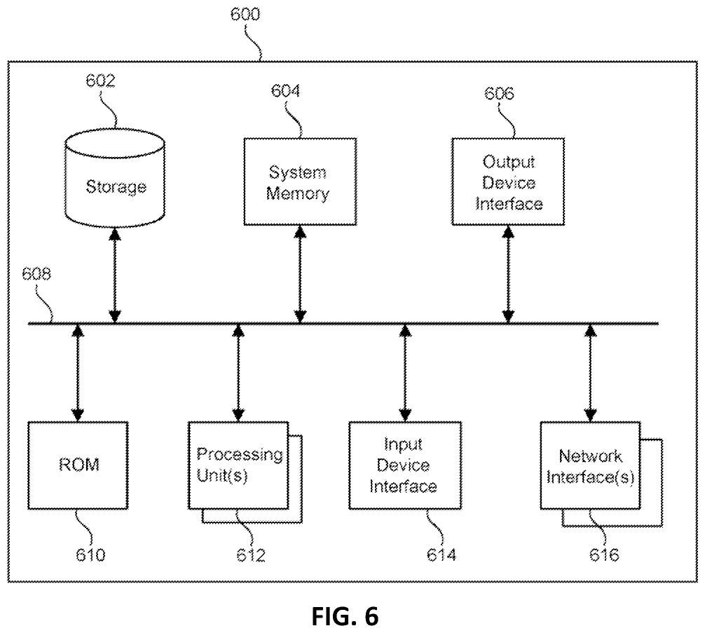

FIG. 6 is a conceptual diagram illustrating an example electronic system 700 for transfusing blood using a syringe pump, according to aspects of the subject technology. Electronic system 600 may be a computing device for execution of software associated with one or more portions or steps of method 500, or components and methods provided by FIGS. 1-5, including but not limited to computing hardware within patient care device 12, or syringe pump 200, and/or any computing devices or associated terminals disclosed herein. In this regard, electronic system 600 may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and methods previously described.

Bus 608 collectively represents all system, peripheral, and chipset buses that communicatively connect the numerous internal devices of electronic system 600. For instance, bus 408 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software, depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A method for transfusing blood using a syringe pump, comprising: connecting a blood supply line to an upstream connection of a check valve; connecting an intravenous transfusion line to a downstream connection of the check valve; connecting a first syringe pump to a second upstream connection of the check valve; activating the first syringe pump to automatically retract and drive a plunger of the first syringe pump, repeatedly, according to a pump rate programmed into the first syringe pump to produce respective negative and positive pressures within the check valve according to an automatic retracting and driving of the plunger, whereby a blood product is withdrawn from the blood supply line, through the upstream connection of the check valve, and into a chamber of a syringe loaded into the first syringe pump during each cycle of the automatic retracting of the plunger, and provided through the intravenous transfusion line from the chamber during each cycle of the automatic driving of the plunger.
Clause 2. The method of Clause 1, further comprising: connecting a drip chamber to the blood supply line; and connecting a blood product container to the drip chamber, wherein the blood product container supplies a blood product to the blood supply line via the drip chamber.
Clause 3. The method of Clause 2, further comprising: connecting a flow control element to the blood supply line; and regulating a flow of the blood product through the blood supply line using the flow control element.
Clause 4. The method of Clause 3, further comprising: connecting a pressure sensor at a location on the intravenous transfusion line downstream of the check valve; monitoring, with the pressure sensor, a real-time delivery pressure associated with flow of the blood product; and automatically adjusting the pump rate based on the real-time delivery pressure.
Clause 5. The method of Clause 4, wherein adjusting the pump rate comprises: increasing a speed at which a plunger of the syringe moves to provide the blood product from the syringe.
Clause 6. The method of any one of Clauses 1 through 5, further comprising: connecting a second check valve to the blood supply line, upstream of the intravenous transfusion line; connecting a second syringe pump to an upstream connection of the second check valve; activating the second syringe pump to automatically retract and drive a plunger of the second syringe pump, repeatedly, wherein the first and second syringe pumps retract and drive their respective plungers according to respective timing patterns offset from each other to deliver the blood product to the intravenous transfusion line.
Clause 7. The method of Clause 6, wherein the first and second syringe pumps are removably connected to a pump control unit by way of respective plugin ports on the pump control unit, wherein the pump control unit comprises a processor and the processor communicates with the first and second syringe pumps via electrical signals communicated through the respective plugin ports.
Clause 8. The method of any one of Clauses 1 through 7, wherein the check valve is a double check valve.
Clause 9. A non-transitory machine-readable storage medium embodying instructions that when executed by a machine, facilitate a machine to perform the method of any one of Clauses 1 through 8.
Clause 10. A system, comprising: one or more processors; and a memory including instructions that, when executed by the one or more processors, cause the one or more processors to perform the method of any one of Clauses 1 through 8.
Clause 11. An infusion device, comprising: the first and second syringe pumps; one or more processors; and a memory including instructions that, when executed by the one or more processors, cause the one or more processors to perform the method of any one of Clauses 1 through 8.

### FURTHER CONSIDERATION

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. A method for transfusing blood using a syringe pump, comprising:
connecting a blood supply line to an upstream connection of a check valve;
connecting an intravenous transfusion line to a downstream connection of the check valve;
connecting a first syringe pump to a second upstream connection of the check valve;
activating the first syringe pump to automatically retract and drive a plunger of the first syringe pump, repeatedly, according to a pump rate programmed into the first syringe pump to produce respective negative and positive pressures within the check valve according to an automatic retracting and driving of the plunger,
whereby a blood product is withdrawn from the blood supply line, through the upstream connection of the check valve, and into a chamber of a syringe loaded into the first syringe pump during each cycle of the automatic retracting of the plunger, and provided through the intravenous transfusion line from the chamber during each cycle of the automatic driving of the plunger.

2. The method of claim 1, further comprising:
connecting a drip chamber to the blood supply line; and
connecting a blood product container to the drip chamber,
wherein the blood product container supplies a blood product to the blood supply line via the drip chamber.

3. The method of claim 2, further comprising:
connecting a flow control element to the blood supply line; and
regulating a flow of the blood product through the blood supply line using the flow control element.

4. The method of claim 3, further comprising:
connecting a pressure sensor at a location on the intravenous transfusion line downstream of the check valve;
monitoring, with the pressure sensor, a real-time delivery pressure associated with flow of the blood product; and
automatically adjusting the pump rate based on the real-time delivery pressure.

5. The method of claim 4, wherein adjusting the pump rate comprises:
increasing a speed at which a plunger of the syringe moves to provide the blood product from the syringe.

6. The method of claim 1, further comprising:
connecting a second check valve to the blood supply line, upstream of the intravenous transfusion line;
connecting a second syringe pump to an upstream connection of the second check valve;
activating the second syringe pump to automatically retract and drive a plunger of the second syringe pump, repeatedly,
wherein the first and second syringe pumps retract and drive their respective plungers according to respective timing patterns offset from each other to deliver the blood product to the intravenous transfusion line.

7. The method of claim 6, wherein the first and second syringe pumps are removably connected to a pump control unit by way of respective plugin ports on the pump control unit, wherein the pump control unit comprises a processor and the processor communicates with the first and second syringe pumps via electrical signals communicated through the respective plugin ports.

8. he method of claim 1, wherein the check valve is a double check valve.

9. A non-transitory machine-readable storage medium embodying instructions that when executed by a machine, facilitate a machine to perform the method of claim 1.

10. A system, comprising:
one or more processors; and
memory including instructions that, when executed by the one or more processors, cause the one or more processors to perform the method of claim 1.

11. An infusion device, comprising:
the first and second syringe pumps;
one or more processors; and
memory including instructions that, when executed by the one or more processors, cause the one or more processors to perform the method of claim 1.
